(19) United States
(12) Patent Application Publication
(10) Pub. No.: US 2026/0325501 A1
(43) Pub. Date: Oct. 22, 2026

(54) AN ORODISPERSIBLE PHARMACEUTICAL COMPOSITION OF FEXOFENADINE AND ITS PROCESS OF PREPARATION

(71) Applicant: NOVUMGEN LIMITED, London (GB)

(72) Inventor: Kamleshkumar PATEL, London (GB)

(21) Appl. No.: 19/106,867

(22) PCT Filed: Aug. 31, 2023
(86) PCT No.: PCT/GB2023/052251

§ 371 Date: Feb. 26, 2025

(30) Foreign Application Priority Data
Aug. 31, 2022 (GB) 2212656.9

(51) Int. Cl. A61K 31/445; A61K 9/00; A61K 9/20

(52) U.S. Cl. CPC A61K 31/445; A61K 9/0056; A61K 9/2009; A61K 9/2013; A61K 9/2027; A61K 9/2054; A61K 9/2059; A61K 9/2095

(57) ABSTRACT

The present invention relates to an orodispersible tablet that can disintegrant in the buccal cavity upon contact with saliva in less than three minutes. The tablet comprises Fexofenadine or pharmaceutically acceptable salts thereof, as well as a combination of excipients that includes at least one disintegrating agent, and at least one diluent agent. The present invention is to offer taste-masking properties and pleasant palatability so that administering the orodispersible tablet is not unpleasant for children, elderly, or unconscious patients, and thus the patient compliance is improved.

### FIELD OF THE INVENTION

The present invention relates to pharmaceutical compositions of fexofenadine. The present invention more particularly relates to orodispersible tablet of fexofenadine or pharmaceutically acceptable salts thereof for oral administration. The present invention also relates to the process of the preparation of the same.

### BACKGROUND OF THE INVENTION

Fexofenadine was disclosed in U.S. Pat. No. 4,254,129. Fexofenadine is a selective H1-antagonist used to treat the symptoms of chronic idiopathic urticaria, and allergic rhinitis. Fexofenadine is a second-generation antihistamine and it is used to treat a variety of allergy symptoms like runny nose, sneezing, itchy, watery eyes, itching of the nose or throat, because it binds to and stabilizes the inactive version of the receptor. Fexofenadine is referred to as a “inverse agonist” of the H1 receptor because it prevents the receptor's activation and consequent downstream effects. Fexofenadine has a potent and selective affinity for H₁ receptors, and there is no evidence that it carries antidopaminergic, antiserotonergic, anticholinergic, sedative, or adrenergic blocking activity. Since fexofenadine cannot cross the blood-brain barrier, it is unlikely to have a substantial impact on the central nervous system.

The IUPAC name of fexofenadine is 2-[4-[1-hydroxy-4-[4-[hydroxy (diphenyl)methyl]piperidin-1-yl]butyl]phenyl]-2-methylpropanoic acid. Fexofenadine is administered as a racemic mixture in which both enantiomers exhibit equivalent antihistamine action.

Fexofenadine is marketed, particularly as an oral tablet and suspension. The dose strengths of fexofenadine that are commercially marketed in 30 mg, 60 mg, 120 mg and 180 mg (depending on the dose). In U.S the dosage strength for suspension is 30 mg/5 ml for oral administration. Fexofenadine is highly efficient when taken orally. Its unpleasant, strong bitter taste after oral administration present a special challenge in the development of oral pharmaceutical formulations of fexofenadine.

The development of orally dispersible tablet has provided a new approach for treating emergency cases, as well as a new technique for oral absorption that solves insoluble drugs simultaneously and is for use by both children and elderly people, especially for gulping down the status of cancer patients undergoing chemotherapy has more advantageous uses, and as a result, found a kind of solution for the quality of life of improving people. Additionally, it has been discovered that the orodispersible tablet of the invention exhibit high stability and physical integrity, for example, during storage, handling, packaging, and similar situations, while maintaining extremely good disintegration performance. The key to manufacturing an orally dispersible tablet is to find the right adjuvant, that is strongly compressible, will disintegrate quickly, and will also have a pleasant mouthfeel.

Fexofenadine is available in racemate or single enantiomer forms, as a free base or as an acid addition salt of the racemate or single enantiomer. Conventionally, the free base form can be converted into an acid salt form, and vice versa. Examples of appropriate salt forms for acid addition include hydrochloride, lactate, and ascorbate, with hydrochloride being the preferred form. The salt hydrochloride of fexofenadine is preferable.

Fexofenadine is 60% to 70% bound to plasma proteins, primarily albumin and α1-acid glycoprotein. Approximately 5% of the total dose of fexofenadine was eliminated by hepatic metabolism. The mean elimination half-life of fexofenadine was 14.4 hours following administration of 60 mg twice daily in healthy adult subjects.

Additionally, some patients, particularly children and the elderly, have trouble swallowing the tablets, even with liquids. It is therefore highly desirable to remedy this issue and create an orodispersible tablet containing fexofenadine that has taste-masking properties, has pleasant palatability, ensures patient comfort during administration, and allows for the acquisition of pharmacokinetic parameters at least bioequivalent to those attained with conventional oral fexofenadine formulations.

U.S. Pat. No. 6,723,348 discloses an orodispersible tablet comprising coated granules of fexofenadine. The tablet consists of a disintegrating agent, a diluent agent, a lubricant and swelling agent, a permeabilizing agent, sweeteners, flavoring agents, and colors in the treatment of seasonal allergic rhinitis. The orodispersible tablets, which are able to disintegrate in the buccal cavity upon contact with saliva, might disintegrate into an easily swallowed suspension in the buccal cavity. The process of preparation of coated granules consists of dry mixing the microcrystals of fexofenadine or its pharmaceutically acceptable salts with a diluent agent or antistatic agent and granulating the mixture obtained in the above step by spraying a solution or suspension with binder and applying a layer over the thus obtained granules by spraying thereon a suspension or solution containing fexofenadine or its pharmaceutically acceptable salts with at least one binder. Thus, coated granules are obtained with a suspension of a coating composition and drying the coating composition will form coated granules. This innovation has drawbacks for coating granules, including a tedious and time-consuming technique that increases formulation costs. The pharmacodynamic properties of drug formulations may be affected by tablet coating, and the process occasionally leads to coating defects such as chipping and cracking etc. and the process for coating granules remains complicated. As a result, highly competent technicians are required to complete the operation.

WO2015/001478A1 discloses oral liquid pharmaceutical composition of fexofenadine or a pharmaceutically acceptable salt thereof for oral administration. The liquid pharmaceutical composition comprises of fexofenadine and/or a pharmaceutically acceptable salt thereof with a pharmaceutically acceptable carrier which is polyethylene glycol or a propylene glycol-based solvent, or a mixture thereof consisting of glycerol and sorbitol as pharmaceutical excipient, stabilizing fexofenadine hydrochloride with flavoring agent and chelating agent such as disodium Ethylenediamine tetra acetic acid (EDTA). This invention is preferably used for infants or children to treat allergies or urticaria. They are bulky, difficult to transport and take up a lot of space. Due to their inherent instability, liquid dosage forms often have shorter shelf lives than their corresponding solid dosage forms. The patient's measurement of the exact volume determines whether the dose is administered appropriately, which increases the chance for variability.

WO2005013987A1 discloses the conventional oral tablet by wet granulation method comprising fexofenadine and/or a pharmaceutically acceptable salt thereof consisting of lactose monohydrate and low substituted hydroxy propyl cellulose. The invention exhibits improved bioavailability as expressed as Cmax. The amount of lactose in pharmaceutical composition is from about 50 wt. % to about 60 wt. % based on the total weight of pharmaceutical composition and the amount of low substituted hydroxy propyl cellulose contains from about 10% to 13% of hydroxy propyl groups. The pharmaceutical composition of fexofenadine or its pharmaceutical acceptable salt thereof is present in amount of from about 10 mg to about 200 mg preferably in 30 to 180 mg and this invention was usefully as antihistamine, antiallergy agent and bronchodilator. The drawbacks of invention disclosed in the patent include tablets are difficult to be swallowed by children, sick, unconscious, and seriously ill patients. Slow breakdown and dissolution of tablets could result in issues with bioavailability.

### SUMMARY OF THE INVENTION

In accordance with present invention, the orodispersible solid pharmaceutical composition for Oral administration is prepared. The solid pharmaceutical composition comprising fexofenadine or pharmaceutically acceptable salts thereof, at least one disintegrant and at least one diluent.

In another embodiment of invention involves process for preparing the solid pharmaceutical composition for Oral administration. The granules prepared by using wet granulation process.

In another embodiment of invention is to provide taste masking properties and present a pleasant palatability such that the administration of the orodispersible tablet is not unpleasant for children, elderly, or unconscious patients, and thus patient compliance is improved.

### OBJECTS OF THE INVENTION

The primary object of the present invention is to provide orodispersible a pharmaceutical composition of fexofenadine or pharmaceutically acceptable salts thereof.

Another object of the present invention is to treat the symptoms of chronic idiopathic urticaria and allergic rhinitis and a variety of allergy symptoms like runny nose, sneezing, itchy, watery eyes, itching of the nose or throat, and it is also used for hay fever, eczema, reactions to insect bites and stings, and some food allergies.

Still another object of the present invention is to provide a solid pharmaceutical composition of fexofenadine or pharmaceutically acceptable salts thereof, suitable for oral administration by the wet granulation.

Still another object of the present invention is to provide a process for preparing the solid pharmaceutical composition of fexofenadine pharmaceutically acceptable salts thereof, for oral administration.

Still another object of the present invention is to provide taste masking properties and a pleasant palatability for children, elderly, or unconscious patients, and thus patient compliance is improved.

Yet another object of the present invention is to provide a stable and uniform oral dispersible pharmaceutical composition of fexofenadine.

Another object of the present invention is to provide fast disintegration of the dosage form as it gets in contact with saliva with a good agreeable mouth feeling.

### DETAILED DESCRIPTION OF THE INVENTION

The Solid pharmaceutical composition of fexofenadine suitable for oral administration is the invention as further described herein.

Drinking water is mostly required for the oral administration of drugs like tablets and capsules, in which some patients experience nuisance in swallowing bulky conventional dosage forms. Orodispersible tablets, a substitute for oral drug delivery system, have been developed in order to reduce dysphagia and increase patient compliance. These tablets are made to dissolve in the mouth without the use of water. Therefore, they are helpful in situations where water is unavailable or forbidden, such as before surgery, during kinetosis, cough bouts brought on by neurological stimulation, or during chest infections. The orodispersible tablets are made using a variety of techniques with the goal of achieving quick disintegration of the dosage form when it comes into contact with saliva while maintaining a pleasant mouthfeel.

Furthermore, the term “orodispersible” as used in the present invention refers to uncoated tablets that can disintegrate in the buccal cavity when comes in contact with saliva. The tablet disintegrates within not more than 3 minutes after oral administration. Orodispersible tablets are also known as orally dispersible and orally disintegrating tablets. These orodispersible tablets can be administered to any patient having difficulty in swallowing.

The preferred embodiment of the invention is an orodispersible solid pharmaceutical composition suitable for oral administration comprising fexofenadine or pharmaceutically acceptable salts thereof, at least one disintegrant, and at least one diluent. When in contact with saliva, these tablets dissolve or disintegrate, releasing the active ingredient and ensuring maximum drug bioavailability compared to conventional dose forms. Super disintegrants, which release the medication in the mouth and increase bioavailability, are added to the dose form to provide this dispersible feature. In one embodiment, the Fexofenadine is present in an amount of 15% w/w to about 45% w/w, preferably in the range from about 20% w/w to about 35% w/w and are administered as oral Solid dosage form.

The term “about,” as and where used in this specification, means ±10% of the mentioned value.

The present invention's solid pharmaceutical composition for oral administration is distinguished by physicochemical properties appropriate for a tablet formulation manufactured by wet granulation, by an adequate active ingredient release rate, and by storage stability attained by using excipients that essentially have no tendency to interact with the active ingredient and have good compressibility properties. To ensure a proper dissolution rate and stability of the finished dosage form, the excipients were carefully chosen. The ultimate objective was to create a stable orodispersible formulation characterized by good taste and rapid disintegration, which leads to greater absorption and high levels of the active ingredient in the systemic circulation.

In one embodiment, a diluent is used in order to enhance the granulation of the fexofenadine or one of its pharmaceutically acceptable salts. Suitable diluent for the present invention can be selected from microcrystalline cellulose, dextrates, dextrose, fructose, sorbitol, starch, pregelatinized maize starch, sucrose, xylitol, maltose, maltodextrin, mannitol and combinations thereof. In the present invention combination of two diluents are used. In the present invention combination of the microcrystalline cellulose and pregelatinized maize starch are preferred as diluent in range of about 20% w/w to about 90% w/w, preferably in the range from about 40% w/w to about 70% w/w. In one embodiment, the microcrystalline cellulose is present in the range from about 20% w/w to about 75% w/w, preferably from about 30% w/w to about 60% w/w and pregelatinized maize starch is present in the range from about 2% w/w to about 20% w/w, preferably from about 4% w/w to about 15% w/w.

In one embodiment, the suitable disintegrant for the present invention is selected from alginic acid, carbon dioxide, carboxymethylcellulose calcium, carboxymethylcellulose Sodium, croscarmellose sodium, guar gum, methylcellulose, polacrilin potassium, poloxamer, Sodium alginate and sodium starch glycolate. Further, the disintegrant can be single or any combination of. A faster disintegration of orodispersible tablets will facilitate an earlier onset of dissolution. The addition of super disintegrants can reduce the disintegration time of tablet. Crosscarmellose sodium is preferred disintegrant for the present invention in the range from about 0.5% w/w to about 10% w/w, preferably in the range from about 1% w/w to about 7.5% w/w. A super disintegrant for use in pharmaceutical formulations, sodium croscarmellose is an internally cross-linked sodium carboxymethylcellulose. Croscarmellose sodium is a super disintegrant that can decrease the disintegration time to less than three minutes. Through the mechanisms of swelling, recovering elastic energy, and capillary action, croscarmellose sodium speeds up the disintegration process (wicking). It is a polymer that resembles fibers and has good flow properties despite its very small size. Its long fiber-like structure can increase the space between the matrix tablet's constituent particles, speeding up the disintegration process. Further, it presents the tendency to absorb water rapidly, so it swells in a significant amount. Therefore, this rapid water absorption by croscarmellose sodium molecules has resulted in a significant increase in the volume of granules, resulting in rapid and uniform disintegration. Croscarmellose sodium incorporated in a pharmaceutical composition facilitates the breakup or disintegration of the content of the tablet into smaller particles that dissolve more rapidly than in the absence of disintegrating agents. Therefore, Croscarmellose is the disintegrant of choice for orodispersible tablets.

As per another embodiment, the solid pharmaceutical composition further comprises a sweetener. The sweetener should be from about 0.5 to 10% w/w, preferably from about 1 to 6.5% w/w. In one embodiment, a suitable sweetener for the present invention is selected from acesulfame potassium, sucralose, cyclamate, saccharin, saccharin sodium, aspartame, or mixtures thereof. The solid pharmaceutical composition of the present invention can be prepared in the presence of acesulfame potassium as a sweetener.

As per one more embodiment, the solid pharmaceutical composition may further comprise a flavoring agent. Flavoring agents may be, for example, mint powder, menthol, orange flavor, vanillin, aspartame, or acesulfame potassium. In the present invention, orange flavor is used for the preparation of orodispersible tablets.

In one embodiment, a suitable binder for the present invention can be selected from the group consisting of alginic acid, carbomer, ethyl cellulose, gelatin, glucose, guar gum, hydroxy ethyl cellulose, methylcellulose, polydextrose, polyethylene oxide, and povidone K30. Preferably, povidone K30 is preferred as a binder for the present invention. It is present in the range from about 0.25% w/w to about 7% w/w, preferably in the range from about 0.5% w/w to about 6% w/w.

Furthermore, as one embodiment of the solid pharmaceutical composition of the present invention, glidant is selected from colloidal silicon dioxide, Talc, calcium silicate, calcium phosphate tribasic and mixtures thereof. Preferably, colloidal silica anhydrous is used as a glidant in the range from about 0.25% w/w to about 7% w/w, preferably in the range from about 0.5% w/w to about 4% w/w.

Furthermore, as one embodiment of the solid pharmaceutical composition of the present invention, the lubricant is selected from boric acid, sodium benzoate, sodium oleate, sodium acetate, sodium lauryl sulphate, magnesium stearate, sodium stearate, calcium stearate, steric acid, waxes or mixtures thereof. Magnesium stearate is preferred as a lubricant for the present invention. It is present in the range from about 0.1% w/w to about 6% w/w, preferably in the range from about 0.5% w/w to about 5% w/w.

Thus, as per one embodiment, the solid pharmaceutical composition of the present invention remains stable at different temperature conditions.

In one more embodiment of the present invention is to provide an orodispersible solid pharmaceutical composition suitable for oral administration comprising fexofenadine, at least one disintegrant, and at least one diluent. In addition, the solid pharmaceutical composition contains at least one pharmaceutically acceptable excipient chosen from the categories of sweetener, flavoring agent, binder, glidant, and lubricant.

Another embodiment of the present invention is to prepare orodispersible dosage forms of fexofenadine by wet granulation process, which is one of the most economical methods. Wet granulation is used mainly to improve the flow and compressibility of powders and to prevent segregation of the blend components. It is used to convert a powder mixture into granules having suitable flow and cohesive properties for tableting. The wet granulation process is preferred to other common manufacturing processes because it improves the hardness of the tablets by reducing friability.

In the preferred embodiment, the disintegrating time of fexofenadine orodispersible tablet is not more than 3 minutes. In order to ensure efficient taste masking and a dissolution profile of the active substance (fexofenadine) such that more than 70% of the fexofenadine is released in 30 minutes, preferably more than 90% is released within 20 minutes.

In the preferred embodiment, the wet granulation process comprising following steps:
Step 1: Weighing all raw materials individually as per the batch formula.
Step 2: Sieving fexofenadine hydrochloride, microcrystalline cellulose 101, pre-gelatinized maize starch, croscarmellose sodium, colloidal anhydrous silica separately through 40 # sieve and acesulfame potassium, orange flavor, and magnesium stearate, separately through 60 # sieve.
Step 3: Dissolving Povidone K30 in Iso Propyl Alcohol with continuous stirring until it gets clear viscous solution.
Step 4: Dry mixing of fexofenadine hydrochloride and microcrystalline cellulose 101 in the rapid mixer granulator.
Step 5: Adding binder solution gradually in dry mixed blend in rapid mixer granulator with continuous mixing.
Step 6: Drying the above granulated blend in a dryer at 50° C.±5° C.
Step 7: Passing dry granules through 24 # sieve and retained granules milled through 1.5 mm screen and pass all blend through 24 # sieve.
Step 8: Mixing of granules with previously shifted Pre-gelatinized maize starch, Croscarmellose Sodium, Colloidal anhydrous silica, Acesulfame Potassium and Orange Flavor in the blender.
Step 9: Mixing of the blend prepared in step 8 with magnesium stearate in a blender.
Step 10: Compressing the resulted mixture into tablet dosage form.

As per one more embodiment of the present invention the solid pharmaceutical composition of fexofenadine is to be used for the treatment of the symptoms of chronic idiopathic urticaria and allergic rhinitis and a variety of allergy symptoms like runny nose, sneezing, itchy, watery eyes, itching of the nose or throat. It is also useful in treatment of hay fever, eczema, reactions to insect bites and stings, and some food allergies.

The invention is further illustrated by the following examples, which are by no means intended to limit the scope of the invention but are given by way of illustration.

### Example 1

| Ingredients | % w/w |
|---|---|
| Fexofenadine hydrochloride | 30.000 |
| Microcrystalline cellulose-102 | 48.835 |
| Povidone K30 | 4.165 |
| Pre-gelatinized maize starch | 7.50 |
| Acesulfame Potassium | 3.00 |
| Colloidal silica anhydrous | 2.00 |
| Orange flavor | 2.00 |
| Magnesium Stearate | 3.00 |
| Total | 100.00 |

### Manufacturing Process:

Step 1: Weighing all raw materials individually as per the batch formula.
Step 2: Mixing of fexofenadine hydrochloride with approximately quantity of the microcrystalline cellulose 102. Sieving mixture through 40 # sieve. Sieving remaining microcrystalline cellulose-102, povidone K30, pre-gelatinized maize starch, colloidal anhydrous separately through 40 # sieve and acesulfame potassium, orange flavor and magnesium stearate through 60 # sieve.
Step 3: Mixing one half of the amount of fexofenadine hydrochloride & microcrystalline cellulose-102, one half of the pre-gelatinized maize starch, povidone K30, colloidal silica anhydrous, acesulfame potassium, orange flavor.
Step 4: Mixing of other half of the amount of fexofenadine hydrochloride & microcrystalline cellulose-102. Then mixing of other half of the pre-gelatinized maize starch, povidone K30, colloidal silica anhydrous, acesulfame potassium and orange flavor in the blender.
Step 5: Mixing of the blend prepared in step 4 with magnesium stearate in a blender.
Step 6: Compressing the resulted mixture into tablet dosage form.

### Observation:

The physical parameters of the blend were found not satisfactory.
Poor flow of blend was observed.

In order to optimize blend flow, change the manufacturing process dry mixing to wet granulation.

| Test Parameters of blend | Result |
|---|---|
| Bulk Density (gm/ml) | 0.552 |
| Tap Density (gm/ml) | 0.747 |
| Carr's Index (%) | 26.10 (poor) |
| Hausner Ratio | 1.35 |

### Example 2

| Ingredients | % w/w |
|---|---|
| Fexofenadine hydrochloride | 30.000 |
| Microcrystalline cellulose-101 | 49.00 |
| Povidone K30 | 3.00 |
| Pre-gelatinized maize starch | 7.500 |
| Acesulfame Potassium | 3.000 |
| Colloidal silica anhydrous | 2.00 |
| Orange flavor | 2.00 |
| Magnesium Stearate | 3.00 |
| Total | 100.00 |

### Manufacturing Process:

Step 1: Weighing all raw materials individually as per the batch formula.
Step 2: Sieving fexofenadine hydrochloride, microcrystalline cellulose 101, pre-gelatinized maize starch, and colloidal anhydrous silica separately through 40 # Sieve and acesulfame potassium, orange flavor and magnesium stearate separately through 60 # sieve.
Step 3: Dissolving Povidone K30 in Iso Propyl Alcohol with continuous stirring until it gets clear viscous solution.
Step 4: Dry mixing of fexofenadine hydrochloride and microcrystalline cellulose 101, in the rapid mixer granulator.
Step 5: Adding binder solution gradually in dry mixed blend in rapid mixer granulator with continuous mixing.
Step 6: Drying the above granulated blend in a dryer at 50° C.±5° C.
Step 7: Passing dry granules through 24 # sieve and retained granules milled through 1.5 mm screen and pass all blend through 24 # sieve.
Step 8: Mixing of granules with previously shifted pre-gelatinized maize starch, colloidal anhydrous silica, acesulfame Potassium and orange flavor in the blender.
Step 9: Mixing of the blend prepared in step 8 with magnesium stearate in a blender.
Step 10: Compressing the resulted mixture into tablet dosage form.

### Observation:

The physical parameters of the tablets were found not satisfactory.
Poor flow of blend was observed due to not proper granules prepared.

In order to minimize poor flow, it was necessary to increase the concentration of binder.

### Example 3

| Ingredients | % w/w |
|---|---|
| Fexofenadine hydrochloride | 30.000 |
| Microcrystalline Cellulose 101 | 48.34 |
| Povidone K30 | 4.17 |
| Isopropyl alcohol | Q.s. |
| Pre-gelatinized maize starch | 7.500 |
| Colloidal silica anhydrous | 2.000 |
| Acesulfame potassium | 3.000 |
| Orange flavor | 2.000 |
| Magnesium Stearate | 3.000 |
| Total | 100.000 |

### Manufacturing Process:

Step 1: Weighing all raw materials individually as per the batch formula.
Step 2: Sieving fexofenadine hydrochloride, microcrystalline cellulose 101, pre-gelatinized maize starch, colloidal anhydrous silica separately through 40 # Sieve and acesulfame potassium, orange flavor and magnesium stearate separately through 60 # sieve.
Step 3: Dissolving Povidone K30 in Iso Propyl Alcohol with continuous stirring until it gets clear viscous solution.
Step 4: Dry mixing of fexofenadine hydrochloride and microcrystalline cellulose 101, in the rapid mixer granulator.
Step 5: Adding binder solution gradually in dry mixed blend in rapid mixer granulator with continuous mixing.
Step 6: Drying the above granulated blend in a dryer at 50° C.±5° C.
Step 7: Passing dry granules through 24 # sieve and retained granules milled through 1.5 mm screen and pass all blend through 24 # sieve.
Step 8: Mixing of granules with previously shifted pre-gelatinized maize starch, colloidal anhydrous silica, acesulfame potassium and orange flavor in the blender.
Step 9: Mixing of the blend prepared in step 8 with magnesium stearate in a blender.
Step 10: Compressing the resulted mixture into tablet dosage form.

### Observation:

The physical parameters of the tablets were found not satisfactory.
Lamination was observed during tablets compression.

In order to minimize lamination issue, it was necessary to decreases the concentration of lubricants.

### Example 4

| Ingredients | % w/w |
|---|---|
| Fexofenadine hydrochloride | 30.000 |
| Microcrystalline cellulose-101 | 50.00 |
| Povidone K30 | 4.17 |
| Isopropyl alcohol | Q.s. |
| Pre-gelatinized maize starch | 7.500 |
| Colloidal silica anhydrous | 2.000 |
| Acesulfame potassium | 3.000 |
| Orange flavor | 2.000 |
| Magnesium Stearate | 1.34 |
| Total | 100.00 |

### Manufacturing Process:

Step 1: Weighing all raw materials individually as per the batch formula.
Step 2: Sieving fexofenadine hydrochloride, microcrystalline cellulose 101, pre-gelatinized maize starch, and colloidal anhydrous silica separately through 40 # sieve and acesulfame potassium, orange flavor and magnesium stearate separately through 60 # sieve.
Step 3: Dissolving Povidone K30 in Iso Propyl Alcohol with continuous stirring until it gets clear viscous solution.
Step 4: Dry mixing of fexofenadine hydrochloride and microcrystalline cellulose 101, in the rapid mixer granulator.
Step 5: Adding binder solution gradually in dry mixed blend in rapid mixer granulator with continuous mixing.
Step 6: Drying the above granulated blend in a dryer at 50° C.±5° C.
Step 7: Passing dry granules through 24 # sieve and retained granules milled through 1.5 mm screen and pass all blend through 24 # sieve.
Step 8: Mixing of granules with previously shifted pre-gelatinized maize starch, colloidal anhydrous silica, acesulfame potassium and orange flavor in the blender.
Step 9: Mixing of the blend prepared in step 8 with magnesium stearate in a blender.
Step 10: Compressing the resulted mixture into tablet dosage form.

### Observation:

The physical parameters of the tablets were found not satisfactory.
Disintegration time was more than 3 minutes

In order to minimize disintegration time, it was necessary to add disintegrant.

### Example 5

| Ingredients | % w/w |
|---|---|
| Fexofenadine Hydrochloride | 30.00 |
| Microcrystalline cellulose 101 | 48.00 |
| Povidone K30 | 4.165 |
| Iso Propyl Alcohol | Q.s. |
| Pre-gelatinized Maize Starch | 7.500 |
| Croscarmellose Sodium | 2.00 |
| Colloidal anhydrous silica | 2.00 |
| Acesulfame Potassium | 3.00 |
| Orange Flavor | 2.00 |
| Magnesium Stearate | 1.34 |
| Total | 100.00 |

### Manufacturing Process:

Step 1: Weighing all raw materials individually as per the batch formula.
Step 2: Sieving fexofenadine hydrochloride, microcrystalline cellulose 101, pre-gelatinized maize starch, croscarmellose sodium and colloidal anhydrous silica separately through 40 # sieve and acesulfame potassium, orange flavor and magnesium stearate separately through 60 # sieve.
Step 3: Dissolving Povidone K30 in Iso Propyl Alcohol with continuous stirring until it gets clear viscous solution.
Step 4: Dry mixing of fexofenadine Hydrochloride and Microcrystalline cellulose 101, in the rapid mixer granulator.
Step 5: Adding binder solution gradually in dry mixed blend in rapid mixer granulator with continuous mixing.
Step 6: Drying the above granulated blend in a dryer at 50° C.±5° C.
Step 7: Passing dry granules through 24 # sieve and retained granules milled through 1.5 mm screen and pass all blend through 24 # sieve.
Step 8: Mixing of granules with previously shifted pre-gelatinized maize starch, croscarmellose sodium, colloidal anhydrous silica, acesulfame potassium and orange flavor in the blender.
Step 9: Mixing of the blend prepared in step 8 with magnesium stearate in a blender.
Step 10: Compressing the resulted mixture into tablet dosage form.

### Observation:

The physical parameters of the tablets were found not satisfactory.
Disintegration time was near to 3 minutes

In order to minimize disintegration time, it was necessary to increase the concentration of disintegrant.

### Example 6

| | | Quantity | Quantity |
|---|---|---|---|
| Ingredients | % w/w | (mg/tablet) | (mg/tablet) |
| Fexofenadine Hydrochloride | 30.00 | 60.00 | 30.00 |
| Microcrystalline cellulose 101 | 46.00 | 92.00 | 46.00 |
| Povidone K30 | 4.165 | 8.33 | 4.165 |
| Iso Propyl Alcohol | Q.s. | Q.s | Q.s. |
| Pre-gelatinized Maize Starch | 7.500 | 15.00 | 7.500 |
| Croscarmellose Sodium | 4.00 | 8.00 | 4.00 |
| Colloidal anhydrous silica | 2.00 | 4.00 | 2.00 |
| Acesulfame Potassium | 3.00 | 6.00 | 3.00 |
| Orange Flavor | 2.00 | 4.00 | 2.00 |
| Magnesium Stearate | 1.34 | 2.67 | 1.34 |
| Total | 100.00 | 200.00 | 100.00 |

### Manufacturing Process:

As per example 5.

### Observation:

All the physical and chemical parameters of fexofenadine hydrochloride 30 mg and 60 mg orodispersible tablets were found to be satisfactory. Tablet is disintegrant within 3 minutes.

IPQC (In Process Quality Control) data of lubricated blend are tabulated below.

| Test Parameters | Result |
|---|---|
| Bulk Density (gm/ml) | 0.376 |
| Tap Density (gm/ml) | 0.486 |
| Carr's Index (%) | 22.47 |
| Hausner Ratio | 1.29 |

### Example 7

The development batches of Example 6 were subjected to stability study 40° C.±2° C./75% RH±5% RH for 1 month. Results are tabulated below.

### Fexofenadine Hydrochloride 30 mg Orodispersible Tablets

| | | | Exp. 6 |
|---|---|---|---|
| | | | (After 1 |
| | | Exp. 6 | month at |
| Test | Specification | (Initial) | 40 °C ± 2 °C) |
| Hardness | 20-90N (Target 50N) | 50N | 52N |
| Friability | NMT 1.0% | 0.12% | 0.15% |
| Disintegration | NMT 3 Minute | 1 min 15 sec | 1 min 20 |
| time | | | sec |
| Assay | 95.0-105.0% of label | 102.4 | 98.3 |
| | claim. | | |
| Dissolution | NLT 75% (Q) labelled | 91.6 | 93.4 |
| | amount of Fexofenadine | | |
| | hydrochloride should be | | |
| | dissolve in 15 minutes. | | |

### Fexofenadine Hydrochloride 60 mg Orodispersible Tablets

| | | | Exp. 6 |
|---|---|---|---|
| | | | (After 1 |
| | | Exp. 6 | month at |
| Test | Specification | (Initial) | 40 °C ± 2 °C) |
| Hardness | 20-90N (Target 50N) | 53N | 47N |
| Friability | NMT 1.0% | 0.09% | 0.11% |
| Disintegration | NMT 3 Minute | 1 min 15 sec | 1 min 4 sec |
| time | | | |
| Assay | 95.0-105.0% of label | 97.3 | 97.2 |
| | claim. | | |
| Dissolution | NLT 75% (Q) labelled | 92.4 | 90.8 |
| | amount of fexofenadine | | |
| | hydrochloride should be | | |
| | dissolve in 15 minutes. | | |

1. An orodispersible tablet of fexofenadine for oral administration comprising;
a) fexofenadine or pharmaceutically acceptable salts thereof;
b) at least one disintegrant present in the range from about 0.5% w/w to about 10% w/w, preferably in the range from about 1% w/w to about 7.5% w/w; and
c) at least one diluent.

2. The orodispersible tablet according to claim 1, wherein the therapeutically effective amount of a fexofenadine or pharmaceutically acceptable salts thereof, is present in an amount of 15% w/w to about 45% w/w, preferably in the range from about 20% w/w to about 35% w/w.

3. The orodispersible tablet according to claim 1, wherein the disintegrant is selected from alginic acid, carbon dioxide, carboxymethylcellulose calcium, carboxymethylcellulose Sodium, croscarmellose sodium, guar gum, methylcellulose, polacrilin potassium, poloxamer, Sodium alginate, and sodium starch glycolate or combination thereof.

4. The orodispersible tablet according to claim 1, wherein the disintegrant is Croscarmellose sodium.

5. The orodispersible tablet according to claim 1, wherein the diluent is selected from microcrystalline cellulose, dextrates, dextrose, fructose, mannitol, Sorbitol, starch, pregelatinized maize starch, Sucrose, Xylitol, maltose, maltodextrin, maltitol, or combinations thereof.

6. The orodispersible tablet according to claim 1, wherein the diluent is the combination of microcrystalline cellulose and pregelatinized maize starch in the range of about 20% w/w to about 90% w/w, preferably in the range from about 40% w/w to about 70% w/w.

7. The orodispersible tablet according to claim 1, further comprising at least one pharmaceutically acceptable excipient selected from lubricant, sweetener, binder, glidants, and flavoring agent.

8. The orodispersible tablet according to claim 7, wherein the lubricant is selected from the group consisting of magnesium Stearate, Sodium Stearyl fumarate, Stearic acid, micronized polyoxyethyleneglycol, Sodium benzoate, and combinations thereof.

9. The orodispersible tablet according to claim 7, wherein the Sweetener is selected from the group consisting of aspartame, potassium acesulfame, Sodium Saccharinate, neo hesperidin dihydrochalcone, Sucralose, monoammonium glycyrrhizinate, and combinations thereof.

10. The orodispersible tablet according to claim 7, wherein the binder is selected from the group consisting alginic acid, carbomer, ethyl cellulose, gelatin, glucose, guar gum, hydroxy ethyl cellulose, methylcellulose, polydextrose, polyethylene oxide, and Povidone K30.

11. The orodispersible tablet according to claim 7, wherein the glidant is selected from colloidal silicon dioxide, Talc, calcium silicate, calcium phosphate tribasic, and combinations thereof.

12. The orodispersible tablet according to claim 7, wherein the flavoring agents is selected from mint powder, menthol, orange flavor, vanillin, aspartame, or acesulfame potassium.

13. The orodispersible tablet according to claim 1, further comprising acesulfame potassium, orange flavor, povidone K30, colloidal silica, and Magnesium stearate.

14. The orodispersible tablet according to claim 1, wherein the orodispersible tablet is manufactured by wet granulation method comprising:
(a) Weighing all raw materials individually as per the batch formula;
(b) Sieving Fexofenadine or pharmaceutically acceptable salts thereof, microcrystalline cellulose 101, pregelatinized maize starch, croscarmellose sodium, colloidal anhydrous silica separately through 40 # sieve and acesulfame potassium, orange flavor, and magnesium stearate, separately through 60 # sieve;
(c) Dissolving Povidone K30 in Iso Propyl Alcohol with continuous stirring until it gets clear viscous solution;
(d) Dry mixing of Fexofenadine or pharmaceutically acceptable salts thereof, and microcrystalline cellulose 101, in the rapid mixer granulator;
(e) Adding binder solution gradually in dry mixed blend in rapid mixer granulator with continuous mixing;
(f) Drying the above granulated blend in a dryer at 50° C.±5° C.;
(g) Passing the dry granules through 24 # sieve and retained granules milled through 1.5 mm screen and pass all blend through 24 # sieve;
(h) Mixing of granules with previously shifted pre-gelatinized maize starch, croscarmellose sodium, colloidal anhydrous silica, acesulfame potassium, and orange flavor in the blender;
(i) Mixing of the blend prepared in step (h) with magnesium stearate in a blender; and
(j) Compressing the resulted mixture into tablet dosage form.

15. The orodispersible tablet according to claim 1, wherein the orodispersible tablet is disintegrant within 3 minutes.

16. The orodispersible tablet according to claim 1, wherein the 70% of the fexofenadine or pharmaceutically acceptable salts thereof, is released within 30 minutes, preferably more than 90% is released within 20 minutes.

17. The orodispersible tablet for oral administration comprising;
a) 15% w/w to about 45% w/w, preferably in the range from about 20% w/w to about 35% w/w of fexofenadine or pharmaceutically acceptable salts thereof;
b) 0.5% w/w to about 10% w/w, preferably in the range from about 1% w/w to about 7.5% w/w of disintegrant;
c) 20% w/w to about 75% w/w, preferably in the range from about 30% w/w to about 60% w/w of diluent;
d) 2% w/w to about 20% w/w, preferably in the range from about 4% w/w to about 15% w/w of diluent;
e) a combination of diluent is present in the range of about 20% w/w to about 90% w/w, preferably in the range from about 40% w/w to about 70% w/w;
f) 0.25% w/w to about 7% w/w, preferably in the range from about 0.5% w/w to about 6% w/w of binder;
g) 0.25% w/w to about 7% w/w, preferably in the range from about 0.5% w/w to about 4% w/w of glidant;
h) 0.1% w/w to about 6% w/w, preferably in the range from about 0.5% w/w to about 5% w/w of lubricant; and
i) 0.5 to 10% w/w, preferably in the range from about 1 to 6.5% w/w of sweetener.

18. The orodispersible tablet according to claim 17, wherein:
a) 15% w/w to about 45% w/w, preferably in the range from about 20% w/w to about 35% w/w of fexofenadine or pharmaceutically acceptable salts thereof;
b) 0.5% w/w to about 10% w/w, preferably in the range from about 1% w/w to about 7.5% w/w of croscarmellose sodium;
c) 20% w/w to about 75% w/w, preferably in the range from about 30% w/w to about 60% w/w of microcrystalline cellulose;
d) 2% w/w to about 20% w/w, preferably in the range from about 4% w/w to about 15% w/w of pregelatinized maize starch;
e) a combination of microcrystalline cellulose and pregelatinized maize starch present in the range of about 20% w/w to about 90% w/w, preferably in the range from about 40% w/w to about 70% w/w;
f) 0.25% w/w to about 7% w/w, preferably in the range from about 0.5% w/w to about 6% w/w of povidone K30;
g) 0.25% w/w to about 7% w/w, preferably in the range from about 0.5% w/w to about 4% w/w of colloidal silica anhydrous;
h) 0.1% w/w to about 6% w/w, preferably in the range from about 0.5% w/w to about 5% w/w of magnesium stearate; and
i) 0.5 to 10% w/w, preferably in the range from about 1 to 6.5% w/w of acesulfame potassium.
